(19) United States
(12) Patent Application Publication
(10) Pub. No.: US 2026/0363816 A1
(43) Pub. Date: Nov. 19, 2026

(54) BIOPSY DEVICE WITH INTEGRATED MARKER

(71) Applicant: BARD PERIPHERAL VASCULAR, INC., Franklin Lakes, NJ (US)

(72) Inventors: Koltin GLASPIE, Chandler, AZ (US); Bryon E. PELZEK, Gold Canyon, AZ (US)

(73) Assignee: BARD PERIPHERAL VASCULAR, INC., Franklin Lakes, NJ (US)

(21) Appl. No.: 19/132,352

(22) PCT Filed: Nov. 30, 2022
(86) PCT No.: PCT/US2022/080689

§ 371 Date: May 22, 2025

(51) Int. Cl. A61B 90/00; A61B 10/02

(52) U.S. Cl. CPC A61B 90/39; A61B 10/0275; A61B 2090/3987

(57) ABSTRACT

A biopsy device generally includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and is moveable between a retaining position and a deploying position. The marker may be retained in a distal portion of the needle lumen by the marker retaining device. Retraction of the marker retaining device from the retaining position to the deploying position is configured to deploy the marker through the sample notch.

### TECHNICAL FIELD

The present specification generally relates to biopsy devices, assemblies, and methods, and, more particularly, to biopsy devices, assemblies, and methods including an integrated marker.

### BACKGROUND

Biopsies are sometimes performed on patients to collect samples of tissue for analysis. A biopsy is generally performed with a biopsy device that is insertable into a patient and configured to collect a tissue sample from the patient while inserted.

In many instances, it may be beneficial to mark the biopsy site with a marker so that the precise location of the biopsy is known for later reference. Markers are generally made from biocompatible materials that are visible under various modes of imaging, such as X-ray. Often, to insert a marker into a patient, a separate applicator containing the marker is inserted into the biopsy device after the tissue sample has been collected. This separate applicator may then be used to deploy the marker into the biopsy site. This process may be cumbersome as it requires handling and inserting a separate applicator containing the marker. Accordingly, it may be beneficial to have a biopsy device with an integrated marker such that the marker is contained within and directly deployable from the biopsy device.

### SUMMARY

Additional features and advantages of the present disclosure will be set forth in the detailed description, which follows, and in part will be apparent to those skilled in the art from that description or recognized by practicing the embodiments described herein, including the detailed description, which follows the claims, as well as the appended drawings.

In one embodiment of the present disclosure, a biopsy device generally includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and is moveable between a retaining position and a deploying position. The marker may be retained in a distal portion of the needle lumen by the marker retaining device. Retraction of the marker retaining device from the retaining position to the deploying position is configured to deploy the marker through the sample notch.

In another embodiment of the present disclosure, a biopsy device generally includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and is rotatable between a retaining position and a deploying position. The marker may be retained in a distal portion of the needle lumen by the marker retaining device. Rotation of the marker retaining device from the retaining position to the deploying position is configured to deploy the marker through the sample notch.

In yet another embodiment of the present disclosure, a method of biopsying tissue includes positioning a biopsy device at a biopsy site. The biopsy device includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and retractable from a retaining position and a deploying position. The marker is retained in a distal portion of the needle lumen by the marker retaining device. The method further includes moving the marker retaining device from the retaining position to the deploying position, thereby deploying the marker from the needle lumen through the sample notch.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein, and together with the description, explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of a biopsy device, according to one or more embodiments shown and described herein;

FIG. 2 schematically depicts a cross-sectional view of the biopsy device of FIG. 1 in a retaining configuration, according to one or more embodiments shown and described herein;

FIG. 3 schematically depicts a cross-sectional view of the biopsy device of FIG. 1 in an intermediate configuration, according to one or more embodiments shown and described herein;

FIG. 4 schematically depicts a cross-sectional view of the biopsy device of FIG. 1 in a deploying configuration, according to one or more embodiments shown and described herein;

FIG. 5 schematically depicts a cross-sectional view a biopsy device in a deploying configuration, according to one or more embodiments shown and described herein;

FIG. 6 schematically depicts a cross-sectional view of a biopsy device in a retaining configuration, according to one or more embodiments shown and described herein;

FIG. 7 schematically depicts a cross-sectional view of the biopsy device of FIG. 6 in an intermediate configuration, according to one or more embodiments shown and described herein; and

FIG. 8 schematically depicts a cross-sectional view of the biopsy device of FIG. 6 in a deploying configuration, according to one or more embodiments shown and described herein.

### DETAILED DESCRIPTION

Reference will now be made in detail to various embodiments of devices, assemblies, and methods, examples of which are illustrated in the accompanying drawings. Whenever possible, the same reference numerals will be used throughout the drawings to refer to the same or like parts. The various figures generally depict a biopsy device and particularly, a biopsy device with a marker contained within, such as through duration of a biopsy procedure, and directly deployable from the biopsy device. The biopsy device generally includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and is moveable between a retaining position and a deploying position. The marker may be retained in a distal portion of the needle lumen by the marker retaining device. Retraction of the marker retaining device from the retaining position to the deploying position is configured to deploy the marker through the sample notch. Accordingly, the marker may be retained within the biopsy device throughout collection of a biopsy sample and deployed directly from the biopsy device. Biopsy procedures are thereby improved by reducing complexity or need of devices used for marker delivery separate from the biopsy device.

Directional terms as used herein—for example up, down, right, left, front, back, top, bottom—are made only with reference to the figures as drawn and are not intended to imply absolute orientation unless otherwise specified. As used herein, “proximal” is used directionally to refer to the direction towards an operator of a biopsy device. Correspondingly, as used herein, “distal” is used directionally to refer to the direction away from the operator of a biopsy device, such as toward to needle tip of the biopsy device.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any device or assembly claim does not actually recite an order or orientation to individual components, or it is not otherwise specifically stated in the claims or description that the steps are to be limited to a specific order, or that a specific order or orientation to components of an device or assembly is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation; and the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

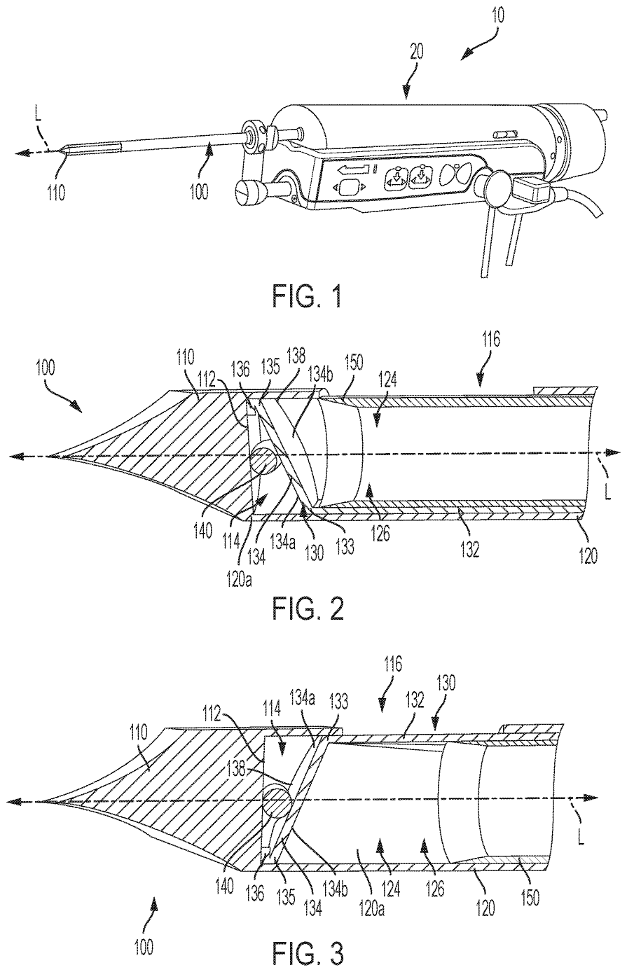

Referring to FIG. 1, a biopsy device 10 is schematically depicted. The biopsy device 10 generally includes a needle 100 and a handle 20. The handle 20 may be configured to house a driver assembly for powering and moving internal components of the biopsy device 10. As will be described herein, the needle 100 extends from the handle 20 to a needle tip 110 and is configured to retrieve a tissue or biopsy sample from a subject.

Referring to FIG. 2, a cross-section of a distal end of the needle 100 is schematically depicted. As shown, the needle 100 generally includes a needle tip 110 and a needle body 120 coupled to the needle tip 110 that extends between the needle tip 110 and the handle 20. The needle tip 110 may be sharp to enable insertion into a subject's tissue to a target location. The needle body 120 may extend about a longitudinal axis L from the needle tip 110. The needle body 120 may be round in a cross-section taken normal to the longitudinal axis L. The needle body 120 may be substantially hollow such that it has an inner surface 120a and a distal wall 112 defining a needle lumen 124. The needle lumen 124 may extend through the needle body 120 along the longitudinal axis L. The needle lumen 124 may be configured to receive a tissue sample.

Referring still to FIG. 2, the needle body 120 may define a sample notch 116 that extends along a portion of the circumference of the needle body 120 and along a portion of the length of the needle body 120 (e.g. in the direction defined by the longitudinal axis L). The sample notch 116 provides a lateral window into the needle lumen 124. As will be described in greater detail herein, the sample notch 116 may be selectively opened to allow for tissue at a biopsy site to move into the needle lumen 124, such as under vacuum pressure, for example. A cutting apparatus 150, as will be described in greater detail below, may then be selectively actuated to extend across the sample notch 116 to sever tissue that has entered the needle lumen 124 through the sample notch 116.

A distal edge of the sample notch 116 may be positioned and proximally spaced from the distal wall 112. In this way, a distal portion 114 of the needle lumen 124 extends distally of the sample notch 116. As will be described in greater detail herein, this distal portion 114 of the needle lumen 124 may house the marker 140 during a biopsy procedure.

The needle 100 may be formed of any medical grade material (e.g., stainless steel, titanium) for performance of a biopsy procedure. In embodiments, the needle tip 110 and the needle body 120 may be integrally formed or may be coupled together through any suitable fastening techniques (e.g., threaded engagement, welding, brazing, etc.).

Still referring to FIG. 2 as noted above, the biopsy device 10 may include a cutting apparatus 150. For example, the cutting apparatus 150 may be a cutting cannula configured to slide between an extended position (depicted in FIG. 2), wherein the cutting apparatus 150 is disposed across the sample notch 116, and a retracted position (FIG. 3), wherein the cutting apparatus 150 is withdrawn proximally from the sample notch 116 to allow for tissue to enter the needle lumen 124 through the sample notch 116. It is noted that the cutting apparatus 150 may be withdrawn in the retracted position only a portion of the distance across the sample notch 116, so as to provide a selectable sample size length.

As depicted, the cutting apparatus 150 may be disposed within the needle lumen 124 so as to be coaxially positioned with the needle 100. In embodiments, the cutting apparatus 150 may, but need not, abut or be in contact with the needle body 120. The cutting apparatus 150 may be substantially round in a cross-section taken perpendicular to the longitudinal axis L and may include a sharp distal edge 152 configured to cut the tissue sample. In some embodiments, the cutting apparatus 150 defines a cutter lumen. In embodiments, the cutter lumen widens or increases in diameter at the sharp distal edge 152 thereby providing a flared opening.

It is noted that while the cutting apparatus 150 is shown as positioned within the needle lumen 124, in some embodiments, the cutting apparatus 150 may instead be disposed outside of the needle lumen 124, such as surrounding the needle body 120. The cutting apparatus 150 may be formed of any medical grade material (e.g., stainless steel, titanium, etc.) suitable for biopsying tissue.

As will be appreciated by those skilled in the art, when the cutting apparatus 150 is in the retracted position and the sample notch 116 is open, the needle 100 may receive a tissue sample (not shown) through the sample notch 116. In embodiments, a vacuum (not shown) may be used to suction the tissue sample into the sample notch 116. While the tissue sample is received within the sample notch 116, the cutting apparatus 150 may be advanced along the longitudinal axis L to the extended position, and the sharp distal edge 152 may cut the tissue sample extending through the sample notch 116 into the needle lumen 124.

Referring still to FIG. 2, positioned within the needle 100 may be a marker 140 housed within the distal portion 114 of the needle lumen 124, so as to be positioned distal to the sample notch 116. As depicted, the marker 140 has a cylindrical shape, though many other shapes are contemplated and possible. The marker 140 may be made from a biocompatible material that is visible during imaging, such as titanium, stainless steel, or Nitinol.

The biopsy device 10 further includes a marker retaining device 130 housed within the needle lumen 124. The marker retaining device 130 is configured to selectively retain the marker 140 in the distal portion 114 of the needle lumen 124. The marker retaining device 130 may be translatable in a longitudinal direction defined by the longitudinal axis L and may be rotatable about the longitudinal axis L. The marker retaining device 130 may include a longitudinally extending portion 132, a marker retaining wall 134 coupled to a distal end 133 of the longitudinally extending portion 132.

The longitudinally extending portion 132 may be a rod, sheet, wire, or the like extending parallel to the longitudinal axis L through the needle lumen 124. For example, and as depicted, the longitudinally extending portion 132. and may be disposed between the cutting apparatus 150 and the needle body 120 and only extend along a portion of the inner diameter of the needle body 120. In embodiments, the longitudinally extending portion 132 may extend to and or within the handle 20 (depicted in FIG. 1), which may include one or more user input devices (e.g., buttons, switch, wheels, motors, etc.) for controlling a position of the longitudinally extending portion 132.

As noted above, the marker retaining wall 134 may be disposed at the distal end 133 of the longitudinally extending portion 132. In embodiments, the marker retaining wall 134 may have a rounded outer edge 138, so as to substantially extend across the diameter of the needle lumen 124 and prevent the marker 140 from exiting the distal portion 114 of the needle lumen 124 prior to deployment. For example, the rounded outer edge 138 may be configured to abut or be positioned close proximate the inner surface 120a of the needle body 120.

As noted above, the marker retaining wall 134 may include a marker contacting portion 136. The marker contacting portion 136 may be disposed at the distal end 135 of the marker retaining wall 134. The marker contacting portion 136 may be a lip or edge raised from a first surface 134a of the marker retaining wall 134. For example, the marker contacting portion 136 may be a rectangular-shaped standoff or flange, though other shapes and other sizes are contemplated and possible. In embodiments, the marker contacting portion 136 may aid in deploying the marker 140 out of the needle lumen 124, as will be described in greater detail below.

In embodiments, the marker retaining device 130 may be made from a shape-memory alloy, such as nickel-titanium, copper-aluminum-nickel, etc., to spring-load the marker retaining device 130, as will be described in greater detail below. In other embodiments, the marker retaining device 130 may be made from a spring material such as spring steel to spring-load the marker retaining device 130.

Referring to FIGS. 2 through 4 in combination, the marker retaining device 130 may be moveable between a retaining position, an intermediate position, and a deploying position. Referring specifically to FIG. 2, the marker retaining device 130 is depicted in the retaining position. In this position, the marker retaining device 130 may be advanced so that the marker 140 is retained in the distal portion 114 of the needle lumen 124. For example, the longitudinally extending portion 132 may be positioned opposite the sample notch 116. In this position, the marker retaining wall 134 may be folded or angled relative to the longitudinally extending portion 132 such that the marker retaining wall 134 divides the distal portion 114 (or a portion thereof) of the needle lumen 124 from a tissue receiving region 126 of the needle lumen 124. In particular, the marker retaining wall 134 may be angled relative to the distal wall 112 and define a marker volume therebetween. In this way, when in the retaining position, the marker retaining wall 134 may have a first surface 134a facing away from the sample notch 116 and a second surface 134b facing the sample notch 116. In embodiments, the marker contacting portion 136 may be in contact with the distal wall 112 and positioned above the marker 140, though other orientations are contemplated and possible.

Accordingly, when the marker retaining device 130 is in the retaining position, it may retain the marker 140 within the distal portion 114 of the needle lumen 124 such that the marker 140 is separated from the sample notch 116, and does not interfere with sample collection.

Referring now to FIG. 3, the marker retaining device 130 is shown in the intermediate position. In this position, the marker retaining device 130 is rotated about the longitudinal axis L relative to the retaining position. Accordingly, the marker retaining device 130, such as the longitudinally extending portion 132, may abut or be positioned adjacent the sample notch 116 in the intermediate position. The marker retaining wall 134 may remain at an angle relative to the longitudinal axis L such that the marker retaining device 130 continues to retain the marker 140 within the distal portion 114 of the needle lumen 124. In this way, when in the intermediate position, the second surface 134b of the marker retaining wall 134 faces away (such as is angled down) from the sample notch 116 and the first surface 134a is angled upward. In the intermediate position, the marker contacting portion 136 may be oriented below the marker 140.

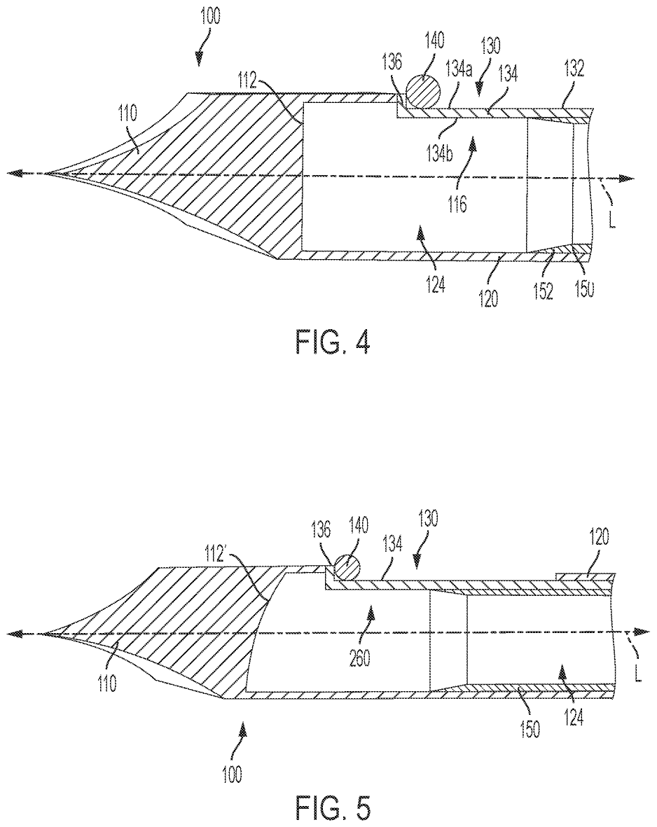

Referring now to FIG. 4, the marker retaining device 130 is shown in the deploying position. In this position, the marker retaining device 130 may be proximally retracted (e.g. in the direction defined by the longitudinal axis L) relative to the intermediate position and the retaining position. As noted above, the marker retaining device 130 may be formed via a shape memory or spring loaded material. Accordingly, as the longitudinally extending portion 132 is withdrawn, the marker retaining wall 134 may be allowed to flatten toward a relaxed orientation to extend in-line (or angled above) the longitudinally extending portion 132. Accordingly, in embodiments the marker retaining wall 134 may be biased to aligned with or extend above the longitudinally extending portion 132, and both the marker retaining wall 134 and the longitudinally extending portion 132 may abut the sample notch 116.

As the marker retaining device 130 is proximally retracted from the intermediate position to the deploying position, the marker retaining wall 134 may continue to move to its relaxed orientation as a result of the bias of the marker retaining wall 134. As the marker retaining device 130 continues to distally retract, the marker contacting portion 136 may maintain contact with the marker 140 to move or “pull” the marker 140 proximally toward the sample notch 116. Accordingly, when the marker retaining device 130 is fully retracted to the deploying position, the marker 140 may be deployed from the sample notch 116 and into the biopsy site of the subject.

As will now be appreciated, in at least some embodiments, a method of using the biopsy device 10 may include positioning the needle 100 at a biopsy site. The marker retaining device 130 may be in the retaining position during positioning of the needle 100 at the biopsy site. During delivery, the cutting apparatus 150 may also be extended distally over the sample notch 116, to prevent unwanted tissue collection through the sample notch 116. While the biopsy device 10 is positioned at the biopsy site, the cutting apparatus 150 may be withdrawn in the proximal direction from the sample notch 116. Once the sample notch 116 is exposed, tissue may enter the sample notch 116. For example, vacuum may be delivered through the sample notch 116, such as via the cutting apparatus 150 and/or the needle lumen 124, to draw tissue into the sample notch 116. The cutting apparatus 150 may then be advanced distally past the sample notch 116 to cut the tissue sample. The sample may be pulled, such as via vacuum through the cutting lumen to a sample collection container. In embodiments, the marker retaining device 130 may remain positioned in the retaining position throughout sample collection.

Once the sample is collected, the method of using the biopsy device 10 may include moving the marker retaining device 130 from the retaining position to the deploying position, thereby deploying the marker 140 from the needle lumen 124 through the sample notch 116. For example, moving the biopsy device 10 from the retaining configuration to the deploying configuration may include rotating the marker retaining device 130 (such as by manipulating the longitudinally extending portion 132 at the handle 20) from the retaining position to the intermediate position (such as depicted in FIG. 3). In the intermediate position, the longitudinally extending portion 132 may be arranged to extend across the sample notch 116, thereby blocking at least a portion of the sample notch 116.

In embodiments, from the intermediate position, the marker retaining device 130 may be withdrawn in the proximal direction from the intermediate position to the deploying position, as depicted in FIG. 4. For example, the longitudinally extending portion 132 may be manipulated at the handle 20 to be pulled in the proximal direction. As the, marker retaining device 130 is withdrawn proximally, the marker retaining wall 134 along with the marker contacting portion 136 may begin to flex toward the sample notch 116, thereby pushing the marker 140 toward and out of the sample notch 116.

Referring now to FIG. 5, in some embodiments, distal wall 112′ of the needle 100 may include a curved or concave surface. For example, the distal wall 112′ may curve toward the needle lumen 124. The distal wall 112′ may accordingly, aid in directing the marker 140 toward the sample notch 116 during movement from the retaining position to the deploying position described above. In particular, the rounded geometry of the distal wall 112′ may prevent the marker 140 from rolling or falling off of the marker retaining device 130 as the marker retaining device 130 is moved from the retaining position to the deploying position.

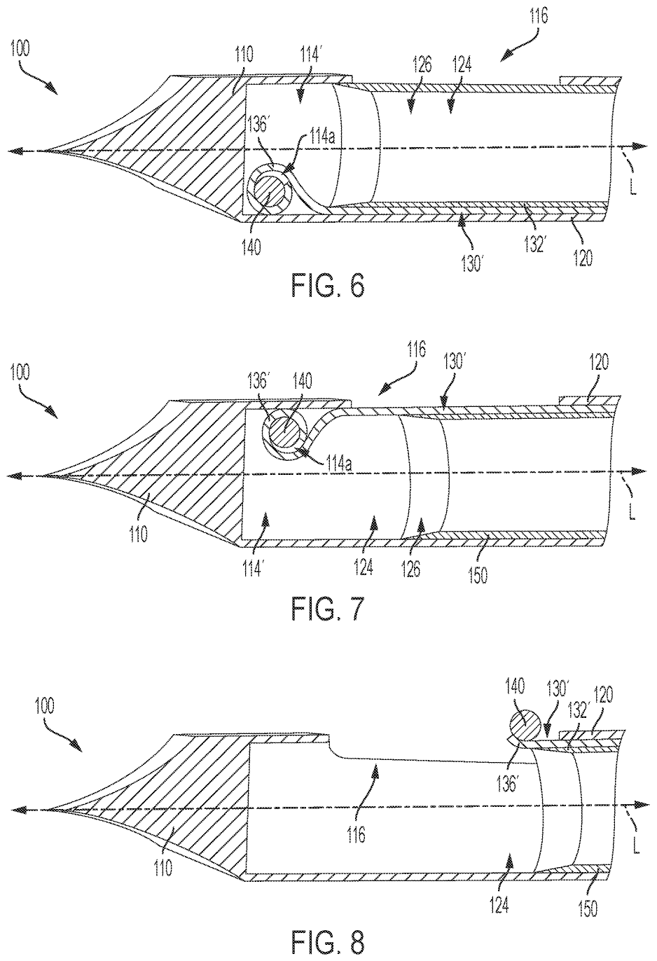

Referring now to FIGS. 6 through 8 in combination, the biopsy device 10 is depicted with an alternative embodiment of a marker retaining device 130′. In the present embodiment, the marker retaining device 130′ may include a longitudinally extending portion 132′ and a marker contacting portion 136′ extending from to the longitudinally extending portion 132′. The marker contacting portion 136′ may be sprung or biased toward a wound geometry such that the marker contacting portion 136′ is wound in about an axis perpendicular to the longitudinally extending axis L. In embodiments, the marker retaining device 130′ may be made from a shape-memory alloy, such as nickel-titanium, copper-aluminum-nickel, etc., to bias the marker contacting portion 136′ of the marker retaining device 130′ to the wound geometry depicted in FIGS. 6 and 7. In other embodiments, the marker retaining device 130′ may be made from a spring material such as spring steel to bias the marker contacting portion 136′ of the marker retaining device 130′.

As depicted in FIG. 6, the marker contacting portion 136′ may be wrapped or wound about the marker 140 when in the retaining position. Accordingly, the marker retaining device 130′ may retain the marker 140 within the distal portion 114′ of the needle lumen 124. In embodiments, the marker retaining device 130′ may separate at least a part 114a of the distal portion 114′ of the needle lumen 124 from the rest of the needle lumen 124. Accordingly, the marker 140 may not be in communication with the sample notch 116 of the needle lumen 124 when the marker retaining device 130′ is in the retaining position.

As depicted in FIGS. 7 and 8, the marker retaining device 130′ may be rotatable and retractable from the retaining position to a deploying position, similar to the embodiment as described above. As depicted particularly in FIG. 7, the marker retaining device 130′ may be rotatable from a position opposite the sample notch 116 to a position abutting the sample notch 116. In the deploying position, as depicted particularly in FIG. 8, the marker retaining device 130′ may be retracted between the cutting apparatus 150 and the needle body 120. As the marker retaining device 130′ is retracted, the marker contacting portion 136′ may unroll to deploy the marker 140 through the sample notch 116. For example, and not as a limitation, the marker contacting portion 136′ may contact an edge of the sample notch 116. The contact between the edge of the sample notch 116 and the marker contacting portion 136′, may cause the marker contacting portion 136′ to unroll as the marker retaining device 130′ is pulled proximally, thereby deploying the marker 140 out of the sample notch 116 to the biopsy site.

Embodiments of the present disclosure may be further described with respect to the following numbered clauses:

1. A biopsy device for marking a location of a tissue biopsy, the biopsy device comprising: a needle comprising a needle tip and a needle body coupled to the needle tip, the needle body defining a needle lumen extending therethrough and a sample notch in communication with the needle lumen; a cutting apparatus operable to cut a tissue sample disposed within the sample notch; a marker retaining device disposed within the needle lumen and moveable between a retaining position (in a distal portion of the needle) and a deploying position (proximal to the retaining position); and a marker retained in a distal portion of the needle lumen by the marker retaining device (when in the retaining position), wherein (the biopsy device/marker retaining device is configured such that) retraction of the marker retaining device from the retaining position to the deploying position deploys the marker through the sample notch.

2. The biopsy device of clause 1, wherein the marker retaining device comprises a marker contacting portion configured to pull the marker in a proximal direction from the distal portion of the needle lumen toward the sample notch (when moving from the retaining position to the deploying position).

3. The biopsy device of any preceding clause, wherein the marker retaining device is spring-loaded to deploy the marker through the sample notch when the marker retaining device is in the deploying position.

4. The biopsy device of any preceding clause, wherein the marker retaining device is rotatable within the needle lumen.

5. The biopsy device of any preceding clause, wherein the marker retaining device is (configured to be) positioned opposite the sample notch when in the retaining position and rotates/is configured to rotate to abut the sample notch when in the deploying position.

6. The biopsy device of any preceding clause, wherein the distal portion of the needle lumen is distal relative to the sample notch.

7. The biopsy device of any preceding clause, wherein the (biopsy device and/or the) marker retaining device is configured to separate/separates the distal portion of the needle lumen from a sample notch when in the retaining position.

8. The biopsy device of any preceding clause, wherein the distal portion of the needle lumen comprises a curved surface.

9. The biopsy device of any preceding clause, wherein the (biopsy device and/or the) marker retaining device is (configured to be) positioned in the retaining position during movement of the cutting apparatus to cut the tissue sample.

10. The biopsy device of any preceding clause, wherein the marker retaining device is configured to be wound about the marker in the retaining position.

In embodiments, the following clauses 11 to 15 can be combined with the biopsy device of clauses 1 to 10.

11. A biopsy device for marking a location of a tissue biopsy, the biopsy device comprising: a needle comprising a needle tip and a needle body coupled to the needle tip, the needle body defining a needle lumen extending therethrough and a sample notch in communication with the needle lumen; a cutting apparatus operable to cut a tissue sample disposed within the sample notch; marker retaining device disposed within the needle lumen and rotatable between a retaining position (in a distal portion of the needle) and a deploying position (proximal to the retaining position); and a marker retained in a distal portion of the needle lumen by the marker retaining device (when in the retaining position), wherein (the biopsy device/marker retaining device is configured such that) rotation of the marker retaining device from the retaining position to the deploying position deploys the marker through the sample notch.

12. The biopsy device of any preceding clause, wherein the marker retaining device is (configured to be) positioned opposite the sample notch when in the retaining position and configured to rotate to abut the sample notch when in the deploying position.

13. The biopsy device of any preceding clause, wherein the (biopsy device and/or the) marker retaining device is configured to separates/separates at least a part of the distal portion of the needle lumen from a tissue receiving region of the needle lumen when in the retaining position.

14. The biopsy device of any preceding clause, wherein the marker retaining device is positioned in the retaining position during movement of the cutting apparatus to cut a tissue sample.

15. The biopsy device of any preceding clause, wherein the distal portion of the needle lumen is distal relative to the sample notch.

In embodiments, clauses 1 to 10 can be combined with the biopsy device of clauses 11 to 15.

16. A method of using a biopsy device, optionally the biopsy device of any of the preceding clauses, the biopsy device comprising: a needle comprising a needle tip and a needle body coupled to the needle tip, the needle body defining a needle lumen extending therethrough and a sample notch in communication with the needle lumen; a cutting apparatus operable to cut a tissue sample disposed within the sample notch; a marker retaining device disposed within the needle lumen and retractable from a retaining position and a deploying position; and a marker retained in a distal portion of the needle lumen by the marker retaining device; and moving the marker retaining device from the retaining position to the deploying position, thereby deploying the marker from the needle lumen through the sample notch.

17. The method of any preceding clause, wherein moving the marker retaining device from the retaining position to the deploying position comprises retracting the marker retaining device.

18. The method of any preceding clause, wherein retracting the marker retaining device moves the marker proximally toward the sample notch.

19. The method of any preceding clause, wherein moving the marker retaining device comprises: rotating the marker retaining device about a longitudinal axis of the needle; and proximally retracting the marker retaining device.

20. The method of any preceding clause, wherein rotating the marker retaining device rotates the marker retaining device from a first position opposite the sample notch to a second position abutting the sample notch.

In view of the above, it should now be understood that at least some embodiments of the present disclosure are directed to a biopsy device that generally includes a needle, a cutting apparatus, a marker, and a marker retaining device. The needle includes a needle tip and a needle body coupled to the needle tip. The needle body defines a needle lumen extending therethrough and a sample notch in communication with the needle lumen. The cutting apparatus is operable to cut a tissue sample disposed within the sample notch. The marker retaining device is disposed within the needle lumen and is moveable between a retaining position and a deploying position. The marker may be retained in a distal portion of the needle lumen by the marker retaining device. Retraction of the marker retaining device from the retaining position to the deploying position is configured to deploy the marker through the sample notch. Accordingly, the marker may be retained within the biopsy device while the tissue sample is cut by the cutting apparatus and may be deployed directly from the biopsy device. Biopsy procedures are thereby improved by reducing complexity or need of devices used for marker delivery separate from the biopsy device.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A biopsy device for marking a location of a tissue biopsy, the biopsy device comprising:
a needle comprising a needle tip and a needle body coupled to the needle tip, the needle body defining a needle lumen extending therethrough and a sample notch in communication with the needle lumen;
a cutting apparatus operable to cut a tissue sample disposed within the sample notch;
a marker retaining device disposed within the needle lumen and moveable between a retaining position and a deploying position; and
a marker retained in a distal portion of the needle lumen by the marker retaining device, wherein retraction of the marker retaining device from the retaining position to the deploying position is configured to deploy the marker through the sample notch.

2. The biopsy device of claim 1, wherein the marker retaining device comprises a marker contacting portion configured to pull the marker in a proximal direction from the distal portion of the needle lumen toward the sample notch.

3. The biopsy device of claim 1, wherein the marker retaining device is spring-loaded to deploy the marker through the sample notch when the marker retaining device is in the deploying position.

4. The biopsy device of claim 1, wherein the marker retaining device is rotatable within the needle lumen.

5. The biopsy device of claim 1, wherein the marker retaining device is positioned opposite the sample notch when in the retaining position and rotates to abut the sample notch when in the deploying position.

6. The biopsy device of claim 1, wherein the distal portion of the needle lumen is distal relative to the sample notch.

7. The biopsy device of claim 1, wherein the marker retaining device separates the distal portion of the needle lumen from the sample notch when in the retaining position.

8. The biopsy device of claim 1, wherein the distal portion of the needle lumen comprises a curved surface.

9. The biopsy device of claim 1, wherein the marker retaining device is positioned in the retaining position during movement of the cutting apparatus to cut the tissue sample.

10. The biopsy device of claim 1, wherein the marker retaining device is wound about the marker in the retaining position.

11. A biopsy device for marking a location of a tissue biopsy, the biopsy device comprising:
a needle comprising a needle tip and a needle body coupled to the needle tip, the needle body defining a needle lumen extending therethrough and a sample notch in communication with the needle lumen;
a cutting apparatus operable to cut a tissue sample disposed within the sample notch;
a marker retaining device disposed within the needle lumen and rotatable between a retaining position and a deploying position; and
a marker retained in a distal portion of the needle lumen by the marker retaining device, wherein rotation of the marker retaining device from the retaining position to the deploying position is configured to deploy the marker through the sample notch.

12. The biopsy device of claim 11, wherein the marker retaining device is positioned opposite the sample notch when in the retaining position and rotates to abut the sample notch when in the deploying position.

13. The biopsy device of claim 11, wherein the marker retaining device separates at least a part of the distal portion of the needle lumen from a tissue receiving region of the needle lumen when in the retaining position.

14. The biopsy device of claim 11, wherein the marker retaining device is positioned in the retaining position during movement of the cutting apparatus to cut the tissue sample.

15. The biopsy device of claim 11, wherein the distal portion of the needle lumen is distal relative to the sample notch.

16. A method of biopsying tissue, the method comprising:
positioning a biopsy device at a biopsy site, the biopsy device comprising:
a needle comprising a needle tip and a needle body coupled to the needle tip, the needle body defining a needle lumen extending therethrough and a sample notch in communication with the needle lumen;
a cutting apparatus operable to cut a tissue sample disposed within the sample notch;
a marker retaining device disposed within the needle lumen and retractable from a retaining position and a deploying position; and
a marker retained in a distal portion of the needle lumen by the marker retaining device; and
moving the marker retaining device from the retaining position to the deploying position, thereby deploying the marker from the needle lumen through the sample notch.

17. The method of claim 16, wherein moving the marker retaining device from the retaining position to the deploying position comprises retracting the marker retaining device.

18. The method of claim 17, wherein retracting the marker retaining device moves the marker proximally toward the sample notch.

19. The method of claim 16, wherein moving the marker retaining device comprises:
rotating the marker retaining device about a longitudinal axis of the needle; and
proximally retracting the marker retaining device.

20. The method of claim 19, wherein rotating the marker retaining device rotates the marker retaining device from a first position opposite the sample notch to a second position abutting the sample notch.
